(19) United States
(12) Patent Application Publication
(10) Pub. No.: US 2025/0302334 A1
(43) Pub. Date: Oct. 2, 2025

(54) SLEEPING POSTURE RECOGNITION METHOD AND SYSTEM BASED ON DEEP NEURAL NETWORK

(71) Applicant: SHENZHEN ONETHIRD SLEEP TECHNOLOGY CO., LTD, Shenzhen (CN)

(72) Inventors: Zhong LIU, Shenzhen (CN); Kuntao LU, Shenzhen (CN); Mingliang SU, Shenzhen (CN)

(21) Appl. No.: 19/237,064

(22) Filed: Jun. 13, 2025

Related U.S. Application Data
(63) Continuation of application No. PCT/CN2023/138516, filed on Dec. 13, 2023.

(30) Foreign Application Priority Data
Dec. 14, 2022 (CN) 202211608610.0

(51) Int. Cl. A61B 5/11; G06N 3/08; G06V 40/20

(52) U.S. Cl. CPC A61B 5/11; G06N 3/08; G06V 40/23

(57) ABSTRACT

Disclosed in the present application are a sleeping posture recognition method and system based on a deep neural network. The method includes the steps of: inputting body pressure sample data into a deep neural network for training learning, to obtain a sleeping posture recognition model; obtaining a two-dimensional body pressure array in real time by using a detection device, the detection device obtaining two-dimensional body pressure analog signal data by means of a pressure sensor array, and converting the two-dimensional body pressure analog data into the two-dimensional body pressure array by means of an A/D conversion module; and transmitting the two-dimensional body pressure array to a server for preprocessing, and inputting the preprocessed two-dimensional body pressure array into the sleeping posture recognition model for recognition.

### CROSS-REFERENCE TO RELATED APPLICATIONS

The present application is a continuation of International Application No. PCT/CN2023/138516, with an international filing date of Dec. 13, 2023, which is based upon and claims priority to Chinese Patent Application No. 202211608610.0 filed on Dec. 14, 2022, the entire contents of all of which are incorporated herein by reference.

### TECHNICAL FIELD

The present application relates to the technical field of sleeping posture recognition, particularly to a sleeping posture recognition method and system based on deep neural network.

### BACKGROUND

Sleep occupies about one-third of a person's life and significantly impacts work, study, and daily life. Sleep is a fundamental need for life and the foundation for maintaining physical and mental health for humans; maintaining good sleep quality plays an extremely important role in the body's self-repair and growth. Studies have shown that sleeping posture is one of the most important factors determining sleep quality, such as sleep stages and sleep difficulties, and is widely used in the medical diagnosis and treatment of sleep disorders.

In prior art, the usual approach is to obtain sleeping posture photos through a camera and judge different sleeping postures by using algorithms for image recognition.

However, people usually cover themselves with blankets during nighttime sleep, which seriously affects the camera's ability to capture data; moreover, obtaining sleeping postures through cameras seriously violates users' privacy.

### SUMMARY

In the prior art, it is difficult to recognize the user's sleeping posture at night through cameras, which also involves a violation of user privacy.

In response to the above issues, a sleeping posture recognition method and system based on CNN deep network are proposed; a pressure sensor array is deployed in a sleeping position on a mattress to obtain body pressure data of different sleeping postures and, then the body pressure sample data is used for training deep neural network to obtain a sleeping posture recognition model, the real-time obtained two-dimensional array of body pressure is used for recognizing sleeping posture, so that the recognition accuracy of sleeping posture is improved and the problem of difficulty in recognizing the user's sleeping posture at night and violation of the user's privacy by cameras in the prior art is addressed.

In the first aspect, a sleeping posture recognition method based on deep neural network, comprising:
Step 100: inputting the body pressure sample data into a deep neural network for training and learning, and obtaining a sleeping posture recognition model;
Step 200: obtaining in real time a two-dimensional array of body pressure using the detection device, wherein the two-dimensional array of body pressure is detected and obtained by the detection device in a sleeping posture on the mattress, the detection device obtains two-dimensional analog signal data of body pressure through a pressure sensor array, and converts the two-dimensional analog data of body pressure into a two-dimensional array of body pressure through an A/D conversion module;
Step 300: transmitting the two-dimensional array of body pressure to a server for preprocessing, inputting the preprocessed two-dimensional array of body pressure into the sleeping posture recognition model for recognition, and outputting the recognition result.

In the first possible embodiment of the sleeping posture recognition method based on deep neural network according to the first aspect of the present application, step 100 comprises:

Step 110: the server preprocesses the received two-dimensional array of body pressure and obtains body pressure sample data.

In the second possible embodiment according to the first embodiment in the first aspect of the present application, step 100 further comprises:
Step 120: setting the number of iterations, weights, and bias values for the deep neural network;
Step 130: inputting the body pressure sample data into the deep neural network and calculating the output error between the expected output and the actual output;
Step 140: comparing the output error with the preset error value and making judgment; and
Step 150: repeating steps 130-140 until the number of iterations is completed or the output error is less than the preset error value, and obtaining the sleeping posture recognition model.

In the third possible embodiment according to the second embodiment in the first aspect of the present application, step 150 comprises the following steps:
Step 151: obtaining the node error and increment for each node in the deep neural network based on the backpropagation of input body pressure sample data;
updating the weight parameters of each layer node of the deep neural network according to the increment.

In the fourth possible embodiment according to the third embodiment in the first aspect of the present application, step 200 comprises:
Step 210: deploying a flexible piezoresistive film in a sleeping position on a mattress;
Step 220: placing the transverse and longitudinal electrodes respectively on both sides of the flexible piezoresistive film to form an M×N array electrode;
the flexible piezoresistive film has an area of Hcm×Lcm to cover partial human torso.

In the fifth possible embodiment according to the fourth embodiment in the first aspect of the present application, step 200 further comprises:
Step 230: converting the detected two-dimensional analog signal data of body pressure into two-dimensional digital signal data of body pressure, and obtaining a two-dimensional array of body pressure;
Step 240: transmitting the two-dimensional array of body pressure to the server.

In the sixth possible embodiment according to the fifth embodiment in the first aspect of the present application, step 300 comprises the following steps:
Step 310: obtaining the first grayscale value of the sleeping posture image corresponding to the maximum body pressure in the two-dimensional array of body pressure;
Step 320: using equation (1) to:

$$Grayscale element = \frac{Single gray scale va1ue}{First grayscale value} \times 255 \qquad ( 1 )$$

obtain a grayscale matrix to reduce noise in the sleeping posture image, and the single grayscale value is the grayscale value in the sleeping posture image corresponding to the element in the two-dimensional array.

In the second aspect, a sleeping posture recognition system based on deep neural network, using the recognition method described in any one of claims 1-7, comprising:
a server;
a detection device;
the detection device is connected to the server for communication;
the detection device, deployed in the sleeping position on a mattress, is used to detect body pressure data of different sleeping postures, and to convert the body pressure data to obtain a two-dimensional array of body pressure;
the server is used to:
preprocess the received two-dimensional array of body pressure, obtain body pressure sample data, and input the body pressure sample data into a deep neural network for training and learning, obtain a sleeping posture recognition model and use the sleeping posture recognition model to recognize real-time preprocessed body pressure data, and output the recognition result;
the detection device comprises:
a pressure sensor array;
and the pressure sensor array is used to obtain two-dimensional analog signal data of body pressure.

In the first possible embodiment according to the sleeping posture recognition system in the second aspect of the present application, the detection device further comprises:
an A/D conversion module;
and the A/D conversion module is electrically connected to the pressure sensor array and is used to convert the two-dimensional analog signal data of the body pressure into a two-dimensional array of body pressure.

In the second possible embodiment according to the sleeping posture recognition system based on deep neural network in the second aspect of the present application, the pressure sensor array comprises:
a flexible piezoresistive film;
a transverse electrode;
a longitudinal electrode;
the transverse and longitudinal electrodes are respectively fitted to both sides of the flexible piezoresistive film, forming an M×N array electrode;
the flexible piezoresistive film has an area of Hcm×Lcm to cover partial human torso.

In the sleeping posture recognition method and system based on deep neural network described in the present application, a sleeping posture recognition method and system based on CNN deep network are proposed, a pressure sensor array is deployed in a sleeping position on a mattress to obtain body pressure data of different sleeping postures and, then the body pressure sample data is used for training the deep neural network to obtain a sleeping posture recognition model, the real-time obtained two-dimensional array of body pressure is used for recognizing sleeping posture, so that the recognition accuracy of sleeping posture is improved and the problem of difficulty in recognizing the user's sleeping posture at night and violation of the user's privacy by cameras in the prior art is addressed.

### BRIEF DESCRIPTION OF THE DRAWINGS

In order to more clearly illustrate the technical solutions of the embodiments of the present application, a brief introduction will be given to the accompanying drawings required for the description of the embodiments. Obviously, the accompanying drawings described below are some embodiments of the present application. Those of ordinary skill in the art can obtain other drawings based on these drawings without creative work.

FIG. 1 is the first schematic diagram of a sleeping posture recognition method based on deep neural network in the present application;

FIG. 2 is the second schematic diagram of a sleeping posture recognition method based on deep neural network in the present application;

FIG. 3 is the third schematic diagram of a sleeping posture recognition method based on deep neural network in the present application;

FIG. 4 is the fourth schematic diagram of a sleeping posture recognition method based on deep neural network in the present application;

FIG. 5 is the fifth schematic diagram of a sleeping posture recognition method based on deep neural network in the present application;

FIG. 6 is the sixth schematic diagram of a sleeping posture recognition method based on deep neural network in the present application;

FIG. 7 is the first schematic diagram of a sleeping posture recognition method based on deep neural network in the present application;

FIG. 8 is the second schematic diagram of a sleeping posture recognition method based on deep neural network in the present application;

FIG. 9 is a schematic diagram of the sensor array structure of a sleeping posture recognition method based on deep neural network in the present application.

### DETAILED DESCRIPTION OF THE EMBODIMENTS

The technical solutions in the embodiments of the present application will be described clearly and completely in conjunction with the accompanying drawings of the embodiments of the present application. Obviously, the described embodiments are only a part of the embodiments of the present application, rather than all the embodiments. Based on the embodiments of the present application, all other embodiments obtained by those of ordinary skill in the art without creative work shall fall within the protection scope of the present application.

In the prior art, it is difficult to recognize the user's sleeping posture at night through cameras, and it also involves the infringement upon user privacy.

A sleeping posture recognition method and system based on CNN deep network are proposed to address the above problem.

### Embodiments of Sleeping Posture Recognition Method

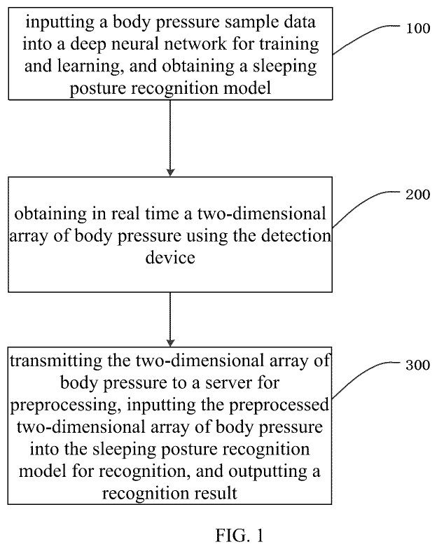

In the first aspect, as shown in FIG. 1, FIG. 1 is the first schematic diagram of a sleeping posture recognition method based on deep neural network in the present application; a sleeping posture recognition method based on deep neural network, comprising: Step 100: inputting a body pressure sample data into a deep neural network for training and learning, and obtaining a sleeping posture recognition model; Step 200: obtaining in real time a two-dimensional array of body pressure using the detection device 10, wherein the two-dimensional array of body pressure is detected and obtained by the detection device 10 in a sleeping posture on the mattress, the detection device 10 obtains two-dimensional analog signal data of body pressure through a pressure sensor array 11, and converts the two-dimensional analog data of body pressure into a two-dimensional array of body pressure through an A/D conversion module 12; Step 300: transmitting the two-dimensional array of body pressure to a server 20 for preprocessing, inputting the preprocessed two-dimensional array of body pressure into the sleeping posture recognition model for recognition, and outputting the recognition result.

There are mainly 9 types of conventional sleeping postures, including fetal sleeping posture on the left side, fetal sleeping posture on the right side, log sleeping posture on the left side, log sleeping posture on the right side, yearner sleeping posture on the left side, yearner sleeping posture on the right side, soldier sleeping posture, starfish sleeping posture, and freefall sleeping posture.

In the embodiment, a sleeping posture recognition method and system based on CNN deep network are proposed, a pressure sensor array 11 is deployed in a sleeping position on a mattress to obtain body pressure data of different sleeping postures and, then the body pressure sample data is used for training deep neural network to obtain a sleeping posture recognition model, the real-time obtained two-dimensional array of body pressure is used for recognizing sleeping posture, so that the recognition accuracy of sleeping posture is improved and the problem of difficulty in recognizing the user's sleeping posture at night and violation of the user's privacy by cameras in the prior art is addressed.

The pressure sensor array 11 can be a flexible piezoresistive film 112 that is deployed in a person's sleeping position inside the mattress to detect the pressure distribution of sleeping posture, and different sleeping postures have different pressure distributions.

The pressure sensor array 11 converts pressure into electrical signals which are initially analog data of body pressure and the analog data of body pressure is converted into digital signals before further processing.

The pressure sensor array 11 may include M×N array electrodes, and piezoresistive conversion elements disposed within the flexible piezoresistive film 112.

In a preferred embodiment, the M×N array electrodes can be assigned specific values, for example, 256×128 array electrodes have 256 transverse electrodes 111 and 128 longitudinal electrodes 113.

Through the A/D conversion module 12, the detected analog data of body pressure is converted into digital signals, and the body pressure data corresponding to a certain sleeping posture is a two-dimensional array of body pressure.

The detection device 10 transmits the two-dimensional array of body pressure to the server 20.

Server 20 also needs to perform image filtering on the images corresponding to the two-dimensional array of body pressure, so as to remove noise from the corresponding images and improve the data accuracy.

In the embodiment, in order to improve the recognition accuracy, the present application constructs a sleeping posture recognition model using preprepared body pressure sample data and deep neural network, and ensures the recognition accuracy through multiple training and learning.

Preferably, step 100 comprises step 110: the server 20 preprocesses the received two-dimensional array of body pressure and obtains body pressure sample data.

Preprocessing is the process where server 20 filters the images corresponding to the received two-dimensional array of body pressure to reduce noise and ensure the accuracy of sample data.

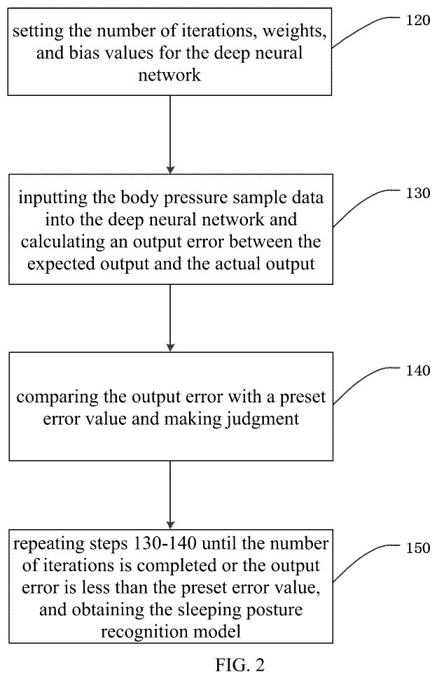

Preferably, as shown in FIG. 2, FIG. 2 is the second schematic diagram of a sleeping posture recognition method based on deep neural network in the present application; step 100 further comprises step 120: setting the number of iterations, weights, and bias values for the deep neural network; step 130: inputting the body pressure sample data into the deep neural network and calculating the output error between the expected output and the actual output; step 140: comparing the output error with the preset error value and making judgment; and step 150: repeating steps 130-140 until the number of iterations is completed or the output error is less than the preset error value, and obtaining the sleeping posture recognition model.

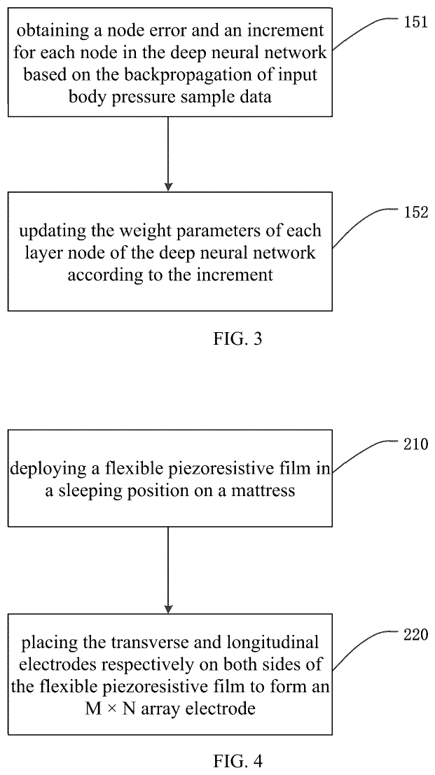

Preferably, as shown in FIG. 3, FIG. 3 is the third schematic diagram of a sleeping posture recognition method based on deep neural network in the present application; step 150 comprises step 151: obtaining the node error and increment for each node in the deep neural network based on the backpropagation of input body pressure sample data; step 152: updating the weight parameters of each layer node of the deep neural network according to the increment.

Predetermined sample data is used and, after multiple iterations or an error smaller than the preset error is output, a sleeping posture recognition model is obtained.

The larger the weight of signal from a deep neural network, the greater the impact it produces, the neural network stores information in the form of weights, and for the purpose of training, weight parameters need to be updated and, in the embodiment of the application, the sleeping posture recognition model uses backpropagation algorithm to update the weight parameters of deep neural network. Specifically, based on the input sample data of body pressure, backpropagation is used to calculate the error and increment for each node and once the error is obtained, the increment of the node can be calculated, and then equation (2) is used to:

$$Δ {W}_{i j} = α {I}_{i} {x}_{j} \qquad ( 2 )$$

$${w}_{i j} + {}_{Δ} {w}_{i j} \to {w}_{i j}$$

Update weight parameters;
wij represents the weight parameter between output node i and input node j, and Δwij is the update amount of weight; xj is the input sample data of corresponding weight, and a is the bias value (0<a≤1), which determines the change amount of weight each time. The increment Ii of node is obtained from the output error of neural network through reverse calculation.

Preferably, as shown in FIG. 4, FIG. 4 is the fourth schematic diagram of a sleeping posture recognition method based on deep neural network in the present application; step 200 comprises step 210: deploying a flexible piezoresistive film 112 in a sleeping position on a mattress; step 220: placing the transverse electrode 111 and longitudinal electrode 113 respectively on both sides of the flexible piezoresistive film 112 to form an M×N array electrode; the flexible piezoresistive film 112 has an area of Hcm×Lcm to cover partial human torso.

The pressure sensor array 11 is mainly divided into three layers, with M horizontal electrodes and N vertical electrodes, and piezoresistive films 112 are disposed between the transverse electrode and longitudinal electrode, providing in M×N pressure detection points.

The area of the flexible piezoresistive film 112 of pressure sensor array 11 can be 100 cm×50 cm, which is suitable for covering the human torso.

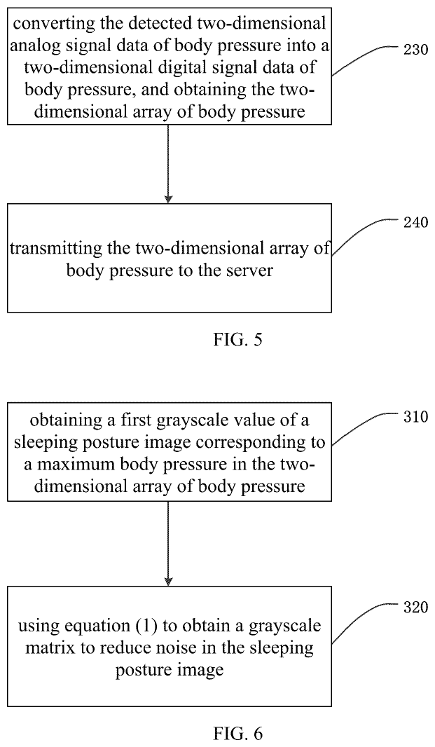

Preferably, as shown in FIG. 5, FIG. 5 is the fifth schematic diagram of a sleeping posture recognition method based on deep neural network in the present application; step 200 comprises step 230: converting the detected two-dimensional analog signal data of body pressure into two-dimensional digital signal data of body pressure, and obtaining a two-dimensional array of body pressure; step 240: transmitting the two-dimensional array of body pressure to the server 20.

Preferably, as shown in FIG. 6, FIG. 6 is the sixth schematic diagram of a sleeping posture recognition method based on deep neural network in the present application; step 300 comprises step 310: obtaining the first grayscale value of the sleeping posture image corresponding to the maximum body pressure in the two-dimensional array of body pressure;

Step 320: using equation (1) to:

$$Grayscale element = \frac{Single gray scale va1ue}{First grayscale value} \times 255 \qquad ( 1 )$$

obtain a grayscale matrix to reduce noise in the sleeping posture image, and the single grayscale value is the grayscale value in the sleeping posture image corresponding to the element in the two-dimensional array.

### Embodiments of Sleeping Posture Recognition System

A sleeping posture recognition system based on deep neural network using the recognition method in the first aspect,

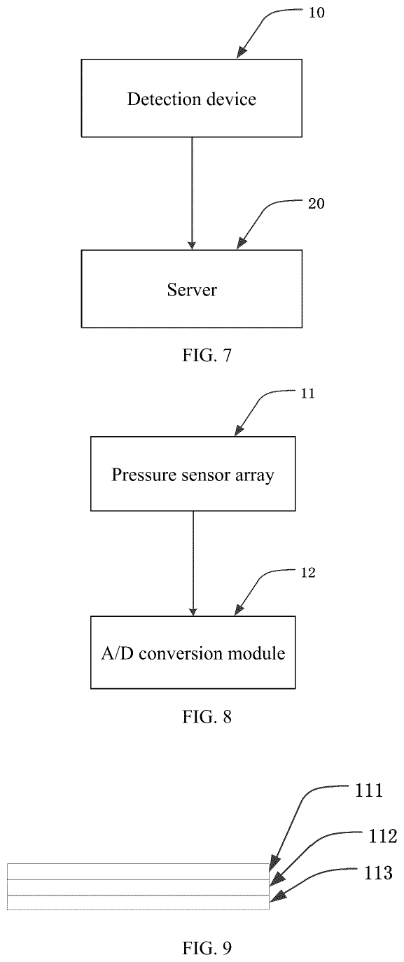

FIG. 7 is the first schematic diagram of a sleeping posture recognition method based on deep neural network in the present application; comprising a server 20 and a detection device 10; the detection device 10 is connected to the server 20 for communication; the detection device 10, deployed in the sleeping position on a mattress, is used to detect body pressure data of different sleeping postures, and to convert the body pressure data to obtain a two-dimensional array of body pressure; the server 20 is used to preprocess the received two-dimensional array of body pressure, obtain body pressure sample data, and input the body pressure sample data into a deep neural network for training and learning, obtain a sleeping posture recognition model and use the sleeping posture recognition model to recognize the real-time preprocessed body pressure data, and output the recognition result; the detection device 10 comprises a pressure sensor array 11.

First, inputting the body pressure sample data into a deep neural network for training and learning, and obtaining a sleeping posture recognition model; obtaining in real time a two-dimensional array of body pressure using a detection device 10, wherein the two-dimensional array of body pressure is detected and obtained by the detection device 10 in a sleeping posture on the mattress, the detection device 10 obtains two-dimensional analog signal data of body pressure through a pressure sensor array 11, and converts the two-dimensional analog data of body pressure into a two-dimensional array of body pressure through an A/D conversion module 12; then transmitting the two-dimensional array of body pressure to a server 20 for preprocessing, inputting the preprocessed two-dimensional array of body pressure into the sleeping posture recognition model for recognition, and outputting the recognition result.

The pressure sensor array 11 is used to obtain two-dimensional analog signal data of body pressure.

In further, as shown in FIG. 8, FIG. 8 is the second schematic diagram of a sleeping posture recognition method based on deep neural network in the present application; detection device 10 also includes an A/D conversion module 12; the A/D conversion module 12 is electrically connected to the pressure sensor array 11 to convert the two-dimensional analog data of body pressure into a two-dimensional array of body pressure.

In further, as shown in FIG. 9, FIG. 9 is a schematic diagram of the sensor array structure of a sleeping posture recognition method based on deep neural network in the present application; the pressure sensor array 11 includes a flexible piezoresistive film 112, a transverse electrode 111, and a longitudinal electrode 113;

The transverse electrode 111 and longitudinal electrode 113 are respectively fitted on both sides of the flexible piezoresistive film 112 to form an M×N array electrode; the flexible piezoresistive film 112 has an area of Hcm×Lcm to cover partial human torso.

The pressure sensor array 11 may include M×N array electrodes, and piezoresistive conversion elements disposed within the flexible piezoresistive film 112. M×N array electrodes can be assigned specific values, for example, 256×128 array electrodes have 256 transverse electrodes 111 and 128 longitudinal electrodes 113.

In the sleeping posture recognition method and system based on deep neural network described in the present application, a pressure sensor array 11 is deployed in a sleeping position on a mattress to obtain body pressure data of different sleeping postures and, then the body pressure sample data is used for training deep neural network to obtain a sleeping posture recognition model, the real-time obtained two-dimensional array of body pressure is used for recognizing sleeping posture, so that the recognition accuracy of sleeping posture is improved and the problem of difficulty in recognizing the user's sleeping posture at night and violation of the user's privacy by cameras in the prior art is addressed.

Only preferred embodiments of the present application are described above and it is not intended to limit the present application. Any modifications, equivalent substitutions, and improvements made within the spirit and principles of the present application should be included in the scope of protection of the present application.

What is claimed is:

1. A sleeping posture recognition method based on deep neural network, comprising:
Step 100: inputting a body pressure sample data into a deep neural network for training and learning, and obtaining a sleeping posture recognition model;
Step 200: obtaining in real time a two-dimensional array of body pressure using the detection device, wherein the two-dimensional array of body pressure is detected and obtained by the detection device in a sleeping posture on the mattress, the detection device obtains a two-dimensional analog signal data of body pressure through a pressure sensor array, and converts the two-dimensional analog data of body pressure into a two-dimensional array of body pressure through an A/D conversion module;
Step 300: transmitting the two-dimensional array of body pressure to a server for preprocessing, inputting the preprocessed two-dimensional array of body pressure into the sleeping posture recognition model for recognition, and outputting a recognition result.

2. The sleeping posture recognition method based on deep neural network of claim 1, wherein step 100 comprises:
Step 110: the server preprocesses the received two-dimensional array of body pressure and obtains the body pressure sample data.

3. The sleeping posture recognition method based on deep neural network of claim 2, characterized in that step 100 further comprises:
Step 120: setting a number of iterations, weights, and bias values for the deep neural network;
Step 130: inputting the body pressure sample data into the deep neural network and calculating an output error between the expected output and the actual output;
Step 140: comparing the output error with a preset error value and making judgment;
Step 150: repeating steps 130-140 until the number of iterations is completed or the output error is less than the preset error value, and obtaining the sleeping posture recognition model.

4. The sleeping posture recognition method based on deep neural network of claim 3, wherein step 150 comprises the following steps:
Step 151: obtaining a node error and an increment for each node in the deep neural network based on the backpropagation of input body pressure sample data;
Step 152: updating the weight parameters of each layer node of the deep neural network according to the increment.

5. The sleeping posture recognition method based on deep neural network of claim 4, wherein step 200 comprises:
Step 210: deploying a flexible piezoresistive film in a sleeping position on a mattress;
Step 220: placing a transverse electrode and a longitudinal electrode respectively on both sides of the flexible piezoresistive film to form an M×N array electrode;
wherein the flexible piezoresistive film has an area of Hcm×Lcm to cover partial human torso.

6. The sleeping posture recognition method based on deep neural network of claim 5, wherein step 200 further comprises:
Step 230: converting the detected two-dimensional analog signal data of body pressure into a two-dimensional digital signal data of body pressure, and obtaining the two-dimensional array of body pressure;
Step 240: transmitting the two-dimensional array of body pressure to the server.

7. The sleeping posture recognition method based on deep neural network of claim 6, wherein step 300 comprises the following steps:
Step 310: obtaining a first grayscale value of a sleeping posture image corresponding to a maximum body pressure in the two-dimensional array of body pressure;
Step 320: using equation (1) to:
$$grayscale element = \frac{single grayscale va1ue}{first grayscale value} \times 255 \qquad ( 1 )$$
obtain a grayscale matrix to reduce noise in the sleeping posture image, and the single grayscale value is the grayscale value in the sleeping posture image corresponding to the element in the two-dimensional array.

8. A sleeping posture recognition system based on deep neural network, using the recognition method of claim 1, comprising:
a server;
a detection device;
the detection device is connected to the server for communication;
the detection device, deployed in a sleeping position on a mattress, is used to detect a body pressure data of different sleeping postures, and to convert the body pressure data to obtain a two-dimensional array of body pressure;
the server is used to:
preprocess the received two-dimensional array of body pressure, obtain a body pressure sample data, and input the body pressure sample data into a deep neural network for training and learning, obtain a sleeping posture recognition model and use the sleeping posture recognition model to recognize the real-time preprocessed body pressure data, and output a recognition result;
the detection device comprises:
a pressure sensor array;
and the pressure sensor array is used to obtain two-dimensional analog signal data of body pressure.

9. The sleeping posture recognition system based on deep neural network of claim 8, wherein the detection device further comprises:
an A/D conversion module;
and the A/D conversion module is electrically connected to the pressure sensor array and is used to convert the two-dimensional analog signal data of the body pressure into the two-dimensional array of body pressure.

10. The sleeping posture recognition system based on deep neural network of claim 9, wherein the pressure sensor array comprises:
a flexible piezoresistive film;
a transverse electrode;
a longitudinal electrode;
the transverse and longitudinal electrodes are respectively fitted to both sides of the flexible piezoresistive film, forming an M×N array electrode;
the flexible piezoresistive film has an area of Hcm×Lcm to cover partial human torso.

11. A sleeping posture recognition system based on deep neural network, using the recognition method of claim 2, comprising:
a server;
a detection device;
the detection device is connected to the server for communication;
the detection device, deployed in a sleeping position on a mattress, is used to detect a body pressure data of different sleeping postures, and to convert the body pressure data to obtain a two-dimensional array of body pressure;
the server is used to:
preprocess the received two-dimensional array of body pressure, obtain a body pressure sample data, and input the body pressure sample data into a deep neural network for training and learning, obtain a sleeping posture recognition model and use the sleeping posture recognition model to recognize the real-time preprocessed body pressure data, and output a recognition result;
the detection device comprises:
a pressure sensor array;
and the pressure sensor array is used to obtain two-dimensional analog signal data of body pressure.
